(19) United States
(12) Patent Application Publication
(10) Pub. No.: US 2025/0212917 A1
(43) Pub. Date: Jul. 3, 2025

(54) Microbial composition and application in reducing biogenic amines

(71) Applicants: Jiangnan University, Wuxi (CN); Jiangnan University (Shaoxing) Industrial Technology Research Institute, Shaoxing (CN)

(72) Inventors: Jian MAO, Wuxi (CN); Shuangping Liu, Wuxi (CN); Shujian Ye, Wuxi (CN)

(21) Appl. No.: 19/000,819

(22) Filed: Dec. 24, 2024

(30) Foreign Application Priority Data
Dec. 27, 2023 (CN) 2023118133859

(51) Int. Cl. A23L 5/20; A23L 7/104; A23L 11/30; C12G 1/00; C12J 1/00; C12N 1/20; C12R 1/225

(52) U.S. Cl. CPC A23L 5/28; A23L 7/104; A23L 11/37; C12G 1/00; C12J 1/00; C12N 1/205; A23V 2400/121; A23V 2400/133; A23V 2400/169; C12R 2001/225

(57) ABSTRACT

The disclosure provides microbial composition and application in reducing biogenic amines, and belongs to the technical field of food processing and manufacturing. The disclosure provides a construction method for an inoculated biogenic amine degrading lactic acid bacteria flora and an application in reducing biogenic amines in cyclic rice steeping and persistently inhibiting invasion of amine producing microorganisms. In the disclosure, a lactic acid bacteria ternary interacting flora that efficiently inhibits invasion of the amine producing microorganisms is constructed. In a rice steeping process in preparation of a brewed product, the lactic acid bacteria ternary interacting flora is inoculated for rice steeping, and a cyclic rice steeping process is improved. The result shows that inoculation of the lactic acid bacteria ternary interacting flora and utilization of the improved rice steeping process may effectively reduce the content of biogenic amines in rice steeping water and completely inhibit invasion of the amine producing microorganisms to realize zero growth of the biogenic amines, thereby creating conditions for the cyclic rice steeping process in mass production in a plant.

### TECHNICAL FIELD

The disclosure relates to microbial composition and application in reducing biogenic amines, and belongs to the technical field of food processing and manufacturing.

### BACKGROUND

Rice steeping is quite important for Huangjiu (Chinese rice wine) brewing, which contributes to the gelatinization of rice when the rice is cooked. Moreover, a produced acid from metabolism provides Saccharomyces with an appropriate acidic environment to guarantee smooth fermentation. However, the metabolic reproduction of amine producing microorganisms in the rice steeping process easily causes sustainable growth of biogenic amines, which affects the safety, quality, and drinking comfort of the Huangjiu. In addition, the rice steeping water contains a lot of nutrient substances such as protein substances, and the colonized metabolism of complex microecological floras makes the rice steeping water smelly, which increases the cost of wastewater treatment and discharge, and causes waste of water resources.

In another patent (publication No. of the patent: CN110747143B), during small-batch production, biogenic amine degrading lactic acid bacteria is inoculated into rice milk and the rice steeping water is recycled, the rice steeping cost is saved, the rice steeping time is shortened, and the content of the biogenic amines is reduced while the quality of the Huangjiu remains unchanged. However, during mass production in a plant, the biogenic amine degrading effect is affected. Infectious microbes left in a complex microecological environment and pipelines of the plant grow and metabolize in the cyclic process. The dominant position of the inoculated biogenic amine degrading lactic acid bacteria is gradually replaced in the cyclic process, which causes massive accumulation of the biogenic amines.

### SUMMARY

The disclosure studies a biogenic amine degrading core microbial flora in a rice steeping water system, and screens and constructs a lactic acid bacteria ternary interacting flora that efficiently inhibits invasion of amine producing microorganisms. The flora is applied to the rice steeping system, so that the acid-producing rate is increased, and the time cost is lowered. On this basis, a cyclic rice steeping process is improved. The disclosure has a relatively strong effect of inhibiting the growth and metabolism of amine producing infectious microbes during the invasion of a lot of amine producing microorganisms in a complex microecological environment and a cyclic rice steeping process, so that recycling of the rice steeping water in mass production in a plant and an application thereof in resisting invasion of the amine producing microorganisms are realized.

In the disclosure, biogenic amine degrading core microorganisms in the rice steeping water are found and assembled in flora to finally provide a ternary microbial interacting flora (including a Lactiplantibacillus plantarum L-53, a Levilactobacillus brevis L-9, and a Latilactobacillus curvatus MG-28). The three strains have been preserved in China Center for Type Culture Collection on Sep. 25, 2023, with the preservation address: Wuhan University, Wuhan, China, and names and preservation No.: L. plantarum L-53 (preservation No.: CCTCC NO: M 20231784), L. brevis L-9 (preservation No.: CCTCC NO: M 20231786), and L. curvatus MG-28 (preservation No.: CCTCC NO: M 20231785).

The disclosure provides a L. plantarum L-53, which has been preserved in China Center for Type Culture Collection on Sep. 25, 2023, with preservation No. CCTCC NO: M 20231784.

The disclosure provides a L. brevis L-9, which has been preserved in China Center for Type Culture Collection on Sep. 25, 2023, with preservation No. CCTCC NO: M 20231786.

The disclosure provides a L. curvatus MG-28, which has been preserved in China Center for Type Culture Collection on Sep. 25, 2023, with preservation No. CCTCC NO: M 20231785.

The disclosure further provides a microbial agent containing the L. plantarum L-53, and/or the L. brevis L-9, and/or the L. curvatus MG-28.

The disclosure further provides an application of the microbial agent in degrading biogenic amines or preparing a biogenic amine degrading product.

The disclosure further provides a microbial agent containing the L. plantarum L-53, the L. brevis L-9, and the L. curvatus MG-28 provided by the disclosure, and the microbial agent may be a liquid preparation or a solid preparation.

The disclosure further provides an application of a ternary interacting flora containing the L. plantarum L-53, the L. brevis L-9, and the L. curvatus MG-28 in degrading biogenic amines.

The disclosure further provides an application of an improved cyclic rice steeping process in resisting the invasion of amine producing microorganisms in a cereal product preparation process by using the ternary interacting flora containing the L. plantarum L-53, the L. brevis L-9, and the L. curvatus MG-28.

The disclosure further provides a composition, containing L. plantarum L-53 or fermentation liquor thereof, L. brevis L-9 or fermentation liquor thereof, and L. curvatus MG-28 or fermentation liquor thereof.

In an embodiment of the disclosure, an adding proportion of the L. plantarum in the composition is at least 23% based on viable count; an adding proportion of the L. brevis in the composition is at least 1-77% based on viable count; and an adding proportion of the L. curvatus in the composition is at least 1-77% based on viable count.

In an embodiment of the disclosure, in the composition, the adding proportion of the L. plantarum L-53 is 6% (v/w) based on mass of a raw material, the adding proportion of the L. brevis L-9 is 2% (v/w) based on mass of the raw material, and the adding proportion of the L. curvatus MG-28 is 1% (v/w) based on mass of the raw material.

The disclosure provides L. plantarum, which has been preserved in China Center for Type Culture Collection on Sep. 25, 2023, with preservation No. CCTCC NO: M 20231784;
the disclosure provides L. brevis, which has been preserved in China Center for Type Culture Collection on Sep. 25, 2023, with preservation No. CCTCC NO: M 20231786; and
the disclosure provides L. curvatus, which has been preserved in China Center for Type Culture Collection on Sep. 25, 2023, with preservation No. CCTCC NO: M 20231785.

In an embodiment of the disclosure, the composition includes, but is not limited to, a microbial agent, a microbial freeze-dried powder, a fermentation additive, and a microbial preparation.

The disclosure further provides an application of the composition in degrading biogenic amines in an environmental system.

The disclosure further provides an application of the composition in preparing a cereal product.

In an implementation method of the disclosure, the cereal product includes any one or more of the following: Huangjiu, Baijiu (Chinese liquor), table vinegar, rice vinegar, cooking wine, rice noodles, rice flour, and soybeans.

In an implementation method of the disclosure, the application includes: in a rice steeping process of the cereal product, inoculating the L. plantarum L-53, the L. brevis L-9, and the L. curvatus MG-28.

In an implementation method of the disclosure, inoculum sizes of the L. plantarum L-53, the L. brevis L-9, and the L. curvatus MG-28 are respectively 1-7%.

In an implementation method of the disclosure, the application includes: recycling rice milk inoculated with the L. plantarum L-53, the L. brevis L-9, and the L. curvatus MG-28 for rice steeping.

In an implementation method of the disclosure, the recycling includes: mixing the rice steeping water added with the composition for rice steeping with clear water in the proportion of 40-60% without inoculating the lactic acid bacterial again for a next round of rice steeping.

In an implementation method of the disclosure, the recycling includes: in the rice steeping process where the L. plantarum L-53, the L. brevis L-9, and the L. curvatus MG-28 are inoculated, replenishing 13%-20% (the ratio of rice to water) of clean water in batches in a fast acid raising period, totally 40%-60% of clean water; performing ventilation at every 8 h and stirring and uniformly mixing the mixture till the acidity is no longer raised for a next round of rice steeping; and repeating the rice steeping steps for cyclic rice steeping.

In an implementation method of the disclosure, prior to performing the next round of cyclic rice steeping, plant rice steeping water which is 10% of total rice steeping water is added to study the ability of the rice steeping water to resist invasion of the amine producing microorganisms.

In an implementation method of the disclosure, the L. plantarum L-53, the L. brevis L-9, the L. curvatus MG-28 are activated before being inoculated. After the L. plantarum L-53, the L. brevis L-9, the L. curvatus MG-28 are subjected to activation and expanded cultivation, a bacteria solution is inoculated into the rice steeping water.

In an implementation method of the disclosure, the L. plantarum, the L. brevis, the L. curvatus are subjected to plate streaking activation, activated 2 times by a liquid MRS and activated by a saccharified liquid of rice, and then the bacteria solution is added into the rice steeping water.

The disclosure further provides a method for inoculating a biogenic amine degrading microbial flora for cyclically steeping rice to inhibit the generation of biogenic amines. The method includes: recycling the rice steeping water added with the composition, where the recycling includes: mixing the rice steeping water added with the composition for rice steeping with clear water in the proportion of 40-60% without inoculating the lactic acid bacteria again for a next round of rice steeping.

In an embodiment of the disclosure, the method includes the following steps:
1) after rice steeping, adding the composition into the rice steeping water, and after static fermentation for 24-36 h in a room temperature condition, replenishing 13%-20% of clean water;
2) after continuous fermentation for 8-24 h in the room temperature condition, replenishing 13%-20% of clean water;
3) after continuous fermentation for 8-24 h in the room temperature condition, replenishing 13%-20% of clean water; and
4) after continuous fermentation for 24-72 h in the room temperature condition, collecting all rice milk for recycling, and stirring and uniformly mixing the rice milk at every 8-24 h during recycling,
where the recycling includes: steeping rice with all rice milk obtained in step 4), where the clean water is not replenished at this time and the lactic acid bacteria is not inoculated again; after rice steeping, performing fermentation for 8-24 h in the room temperature condition and replenishing 13%-20% of clean water; performing continuous fermentation for 8-24 h in the room temperature condition and replenishing 13%-20% of clean water; performing continuous fermentation for 8-24 h in the room temperature condition and replenishing 13%-20% of clean water; performing continuous fermentation for 8-24 h in the room temperature condition; and collecting all rice milk for the next round of rice steeping, and stirring and uniformly mixing the rice milk at every 8-24 h during recycling.

In an embodiment of the disclosure, the method includes: after rice steeping, adding the composition; after static fermentation for 24 h in a room temperature condition, replenishing 13%-20% of clean water; after continuous fermentation for 16 h in the room temperature condition, replenishing 13%-20% of clean water; after continuous fermentation for 8 h in the room temperature condition, replenishing 13%-20% of clean water; after continuous fermentation for 48 h in the room temperature condition, collecting all rice milk for recycling, and stirring and uniformly mixing the rice milk at every 8 h during recycling, where the recycling includes: steeping rice with all rice milk, where the clean water is not replenished and the lactic acid bacteria is not inoculated again; performing fermentation for 8 h in the room temperature condition and replenishing 13%-20% of clean water; performing continuous fermentation for 8 h in the room temperature condition and replenishing 13%-20% of clean water; performing continuous fermentation for 8 h in the room temperature condition and replenishing 13%-20% of clean water; performing continuous fermentation for 24 h in the room temperature condition; and collecting all rice milk for the next round of rice steeping, and stirring and uniformly mixing the rice milk at every 8 h during recycling.

In an embodiment of the disclosure, the L. plantarum, the L. brevis, and the L. curvatus are activated before being inoculated, and the activating method is step-by-step extended cultivation according to the order of a solid plate, a triangular flask, and a cultivating pot, with specific operations as follows: performing first activation on the lactic acid bacteria on a solid MRS culture medium, picking a single colony and culturing the single colony in a liquid MRS culture medium at 28-37° C. for 12-24 h, inoculating the lactic acid bacteria into a saccharified liquid with an inoculum size of 1%, and culturing the lactic acid bacteria at 28-37° C. for 36-72 h to complete third activation.

In an embodiment of the disclosure, the lactic acid bacteria are activated before being inoculated, and the activating method is step-by-step extended cultivation according to the order of a solid plate, a triangular flask, and a cultivating pot, with specific operations as follows: performing first activation on the lactic acid bacteria on a solid MRS culture medium, picking a single colony and culturing the single colony in a liquid MRS culture medium at 37° C. for 24 h, inoculating the lactic acid bacteria into a saccharified liquid with an inoculum size of 1%, and culturing the lactic acid bacteria at 37° C. for 48 h to complete third activation.

In an embodiment of the disclosure, in the composition, the adding proportion of the L. plantarum L-53 is 6% (v/w) based on mass of a raw material, the adding proportion of the L. brevis L-9 is 2% (v/w) based on mass of the raw material, and the adding proportion of the L. curvatus MG-28 is 1% (v/w) based on mass of the raw material.

The disclosure provides a method for constructing a biogenic amine degrading microbial flora, which performs screening and recombination to obtain a ternary microbial interacting flora with a biogenic amine degrading ability and is applied to resisting invasion of the amine producing microorganisms in a rice steeping process of the cereal product. After the ternary microbial interacting flora is inoculated into the Huangjiu rice steeping water, expanded cultivation of the rice steeping water is performed, and before the next round of cyclic rice steeping is performed, the plant rice steeping water is added. In the cyclic rice steeping process, in the case of invasion of the amine producing microorganisms, zero growth of the biogenic amines is realized. The disclosure creates conditions for the application of the cyclic rice steeping process in a complex microecological environment of the plant and in the case of infectious microbes left in pipelines of a rice steeping pot.

### Beneficial Effects

(1) The ternary interacting flora containing the L. plantarum L-53, the L. brevis L-9, and the L. curvatus MG-28 provided by the disclosure has the ability to not generate but degrade biogenic amines and may accelerate the acid-producing rate; and only 4 days are needed for first-time rice steeping, only 2 days are needed for cyclic rice steeping, and compared with conventional rice steeping, the time is shortened by 3-5 days.

(2) Unpleasant odor and “white foam” of rice steeping are also eliminated in the disclosure while rapid rice steeping is realized, so that the rice milk has a pleasant sour, and the wine brewed by the rice steeping process reaches a superior quality.

(3) By using the method provided by the disclosure, which includes: after inoculating the L. plantarum L-53, the L. brevis L-9, and the L. curvatus MG-28 into the rice steeping water, performing static fermentation in the room temperature condition, replenishing 13%-20% of clean water at the 24^{th} hour, the 40^{th} hour, and the 48^{th} hour, performing ventilation at every 8 h, stirring and uniformly mixing the mixture, and performing persistent fermentation for 96 h. After rice steeping is finished, the content of the biogenic amines is 10 mg/L or less, which, compared with that in the conventional rice steeping, is decreased by 91.15% or more. After the first time rice steeping is finished, all rice steeping water is collected for cyclic rice steeping. Before each cyclic rice steeping, 10% of plant rice steeping water is added to simulate the interference process of the amine producing infectious microbes in the plant rice steeping environment, 13%-20% of clean water is respectively replenished at the 8^{th} hour, the 16^{th} hour, and the 24^{th} hour, ventilation is performed at every 8 h, and stirring and uniformly mixing are performed.

In the case of adding the amine producing infectious microbes, the disclosure fully inhibits the growth and metabolism of the infectious microbes, and the content of the biogenic amines within 4 cycles is “zero growth”.

In another patent, in a process of simulating the infectious microbe interference resisting process in the plant environment (publication No.: CN110747143B), the biogenic amines of the culture rise continuously. The biogenic amines in the 4^{th} cycle increase to 59.01±3.02 mg/L, and the content of the biogenic amines is 11 times that in the disclosure. It may be seen that the effect of the disclosure is far superior to that of the above disclosed patent document.

(4) Compared with conventional rice steeping, the method provided in the disclosure may shorten the rice steeping time by 40%-70% and reduce the content of the biogenic amines by 91.15% or more, and achieve zero discharge of wastewater. Moreover, the disclosure has relatively strong abilities to resist infectious microbe interference and invasion of the amine producing microorganisms. In the case of interference of the infectious microbe in the cyclic process, growth of the amine producing microorganisms is completely inhibited, and “zero growth” of the biogenic amines is realized, thereby creating feasibility to apply the cyclic rice steeping process in mass production in the plant.

### BRIEF DESCRIPTION OF FIGURES

FIG. 1: a content change of 30 groups of biogenic amines in each generation in manual enriching and screening.

FIG. 2: a structural composition of bacterial floras in each generation rice steeping water in manual enriching and screening.

FIG. 3: a correlation heatmap of bacterial changes and physical and chemical indicators in different batches of rice milk (p<0.05).

FIG. 4: a correlation heatmap of bacterial changes and physical and chemical indicators in high and low biogenic amine groups (p<0.05).

FIG. 5: redundancy analysis of microbial flora structures and biogenic amines.

FIG. 6: inhibition of different inoculating types on biogenic amine producing microorganisms.

FIG. 7: inhibition of different inoculating types on biogenic amine producing microorganisms during cyclic rice steeping.

FIG. 8: an inhibitory effect of a ternary lactic acid bacteria interacting flora on biogenic amines.

FIG. 9: changes of acidity of an optimized and improved cyclic rice steeping process.

### PRESERVATION OF BIOLOGICAL MATERIALS

A L. plantarum L-53, a taxonomic name of which is L. plantarum L-53, which has been preserved in China Center for Type Culture Collection on Sep. 25, 2023, with preservation No. CCTCC NO: M 20231784 and the preservation address: Wuhan University, Wuhan, China.

A Lactiplantibacillus brevis L-9, a taxonomic name of which is L. brevis L-9, which has been preserved in China Center for Type Culture Collection on Sep. 25, 2023, with preservation No. CCTCC NO: M 20231786 and the preservation address: Wuhan University, Wuhan, China.

A Lactiplantibacillus curvatus MG-28, a taxonomic name of which is L. curvatus MG-28, which has been preserved in China Center for Type Culture Collection on Sep. 25, 2023, with preservation No. CCTCC NO: M 20231785 and the preservation address: Wuhan University, Wuhan, China.

### DETAILED DESCRIPTION

### Technical Terms

The term “bacterial strain” used herein refers to microorganisms of a special species with joint features. Unless otherwise indicated, the terms “bacterial strain” and “cell” used herein may be interchangeably used.

The term “plate” used herein refers to a plate culture medium, which is the most common solid culture medium form for obtaining a pure culture of microorganisms. It is a culture medium solid plane formed by the cooled and condensed solid culture medium in a sterile culture dish, which is usually abbreviated as a culture plate or a plate.

The term “culture medium” used herein refers to a culture medium, which includes chemical elements required by the growth of the microorganisms, together with at least one carbon source and one nitrogen source.

The term “culture” used herein refers to the culture of the microorganisms for a period of time till an expected goal is achieved.

The term “fermentation liquor” used herein refers to inoculating a microbial culture into a liquid culture medium, where after culture for a period of time, the microorganisms synthesize thalli and secretory products by using nutrient components in the culture medium, and the liquid metabolized by the microorganisms is called the fermentation liquor.

The strain provided by the disclosure further includes mutants, variants, and/or offsprings of the L. plantarum strain, the L. brevis strain, and the L. curvatus strain.

The “mutants” refer to any microorganisms generated by modifying the L. plantarum strain, the L. brevis strain, or the L. curvatus strain. For example, the mutants may be microorganisms generated by gene modification of the L. plantarum strain, the L. brevis strain, or the L. curvatus strain. The “variants” refer to naturally existing microorganisms originated from parental L. plantarum strain, L. brevis strain, or L. curvatus strain. For example, the variants may be microorganisms generated by the L. plantarum strain, the L. brevis strain, or the L. curvatus strain adapted to a special cell culture condition. The “offsprings” refer to any microorganisms generated the parental L. plantarum strain or the mutants thereof (the L. brevis strain or the mutants thereof, the L. curvatus or the mutants thereof) and the variants after reproduction or proliferation, where the offspring strains themselves may be recognized as strains the same as or essentially same as the parental strains. It may be understood that in view of a vegetative propagation process, the offspring strains are nearly completely the same as the parental L. plantarum strain, L. brevis strain, or L. curvatus strain in terms of gene. Therefore, the offspring strains are completely the same as the parental strains in terms of gene, and may be regarded as “clones” of the parental strains. Or, the offspring strains are completely the same as the parental strains essentially.

The term “microbial flora” used herein refers to an aggregate formed by tiny microorganisms in an ecological system. This concept covers various microorganisms, including bacteria, fungi, archaea, and protozoa, which jointly live in a special environment and form an ecological system by means of interactions. Features of the microbial flora are determined by types, quantities, diversities, and interactions of these microorganisms. The microbial flora herein refers to various lactic acid bacteria, acetic acid bacteria, Weissella cibaria and the like colonized in a rice steeping water ecological system, which interact to jointly control fermentation of the rice steeping water, generation of the biogenic amines, and changes of the physical and chemical indicators.

The term “biogenic amine (BA)” used herein is a non-volatile low molecular nitrogen-containing organic base, which is mainly generated by decarboxylating a free amino acid substrate under the action of amino acid decarboxylase, and moreover, CO₂ is generated. Some biogenic amines may be generated by amination and transamination of aldehyde and ketones. This path is less common. Common biogenic amines mainly include putrescine, tyramine, cadaverine, histamine, phenylethylamine, tryptamine, spermine, and spermidine, which are respectively generated by decarboxylating ornithine, tyrosine, lysine, histidine, phenylalanine, tryptophan, and arginine. Putrescine is formed by arginine and agmatine precursors as well. Generation and consumption of the biogenic amines may be divided into three steps: free amino acids generate the biogenic amines under the action of the amino acid decarboxylase; the biogenic amines are oxidized by amine oxidase (reacted with oxygen and water) to generate an aldehyde substance. The aldehyde substance is finally oxidized into carboxylic acid, which may be utilized by the body for energy production or direct discharge.

The term “composition” used herein refers to one or more microbial preparations or microbial agents or fermentation additives or other products including the L. plantarum or the fermentation liquor thereof, the L. brevis or the fermentation liquor thereof, the L. curvatus or the fermentation liquor thereof.

### The Culture Media Involved in the Examples Below are as Follows:

MRS culture medium formula: 10.0 g/L peptone, 10.0 g/L beef extract, 5.0 g/L yeast extract, 2.0 g/L diammonium hydrogen citrate, 20.0 g/L glucose, 1.0 mL/L Tween 80, 5.0 g/L sodium acetate, 2.0 g/L dipotassium phosphate, 0.58 g/L magnesium sulfate, 0.25 g/L manganese sulfate, 20.0 g/L agar (added into the solid MRS culture medium); pH 6.5.

Saccharified liquid: glutinous rice, malt flour, and water are mixed at 1:0.2:4 (m/m/v), 1‰ of a saccharifying enzyme and a liquifying enzyme and 15% of wheat koji are added, the mixture is insulated and saccharified at 60° C. for 4 h and stirred once at every 1 h, and after saccharification, the mixture is sterilized at 115° C. for 20 min.

### Detection Methods Involved in the Examples Below are as Follows:

Determination of the biogenic amines refers to National Food Safety Standard GB 5009.208-2016—Determination of Biogenic Amines in Food.

Total acid determination refers to a national standard GB/T 13662-2018 Huangjiu.

Plant rice steeping water involved in the examples below is taken from Zhejiang Guyuelongshan Shaoxing Wine Co. Ltd., and a preparation method is as follows:

10 t of glutinous rice is put in a jug in a plant of Zhejiang Guyuelongshan Shaoxing Wine Co. Ltd., with the ratio of rice to water being 1:1.2, the mixture stands still for 7 days, and rice milk is collected.

### Physical and Chemical Parameters of the Plant Rice Steeping Water in the Examples Below are as Follows:

Plant rice steeping water: the total acid is 11.23±1.13 g/L, the biogenic amines are 313.41±5.16 mg/L, the COD content is 47144±2.31 mg/L, and the BOD5 content is 32592±4.12 mg/L. The microbial flora structure of the plant rice steeping water obtained by high-throughput sequencing is analyzed below: it is mainly based on Lactobacillus (30.81%), Lactococcus (57.06%), and Weissella (9.12%).

The objective of adding the plant rice steeping water into the rice steeping system is to add the amine producing bacteria in the rice steeping system to study the inhibiting ability of the strain provided by the disclosure on the amine producing microorganisms in the plant rice steeping water.

The L. plantarum L1901 involved in the examples below is recorded in a Chinese patent for invention with publication No. CN110747143B.

### Example 1: Prediction of a Biogenic Amine Degrading Core Microbial Flora in Rice Steeping Water

### 1. Artificial Enrichment Experiment

30 sterilized centrifuge tubes were taken, 10 g of radiated rice (25 kGy) and 20 ml of sterile water were added, 200 μL of plant rice steeping water was inoculated into each centrifuge tube, and the centrifuge tube was sealed with a sterilized sealing film for culture for 2 d. After culture was finished, the content of the biogenic amines was determined for each group. Three groups with the lowest content of the biogenic amines were selected and gathered for inoculating a next generation. The same inoculating step was repeated for each generation, and the process was repeated for n generations. It played a role of selectively enriching the biogenic amine degrading microorganisms.

### 2. Detection of the Microbial Flora Structure

The above steps were performed for 15 generations, the 3 groups with the lowest content of the biogenic amines in each generation and difference value points with high content of the biogenic amines in each generation were selected for high-throughput sequencing, redundancy analysis was performed and Spearman correlation coefficients were calculated in combination with the content of the biogenic amines, to explore the core microbial flora reducing the biogenic amines.

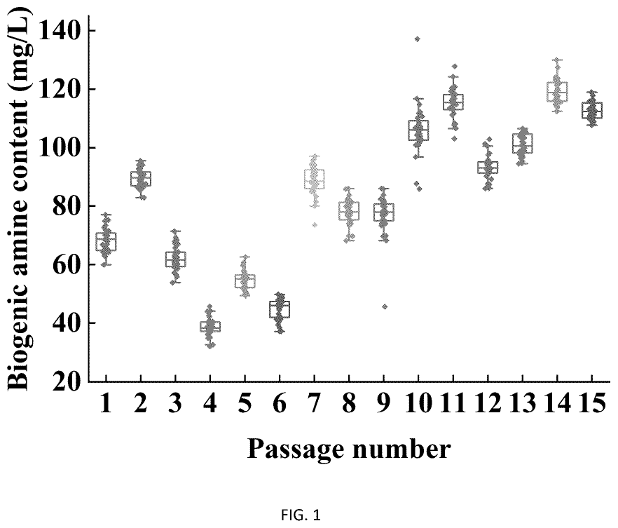

In the artificial selective enrichment experiment, the content change of the biogenic amines was shown in FIG. 1, the content of the biogenic amines was not persistently reduced according to an expected result and was generally in a trend of decrease first and then increase. The lowest average content of the biogenic amines in the 4^{th} group would reach 31.97 mg/L, and the highest content of the biogenic amines in the 14^{th} group would reach 119.35 mg/L. Therefore, the flora structures of 3 groups with the lowest content of the biogenic amines in each group and abnormal value points of 3 groups with the highest content of the biogenic amines in the 10^{th}, 11^{th}, and 14^{th} generations were determined, and in combination with the biogenic amine determination result, the core microorganisms causing the biogenic amines to rise or reduce were analyzed.

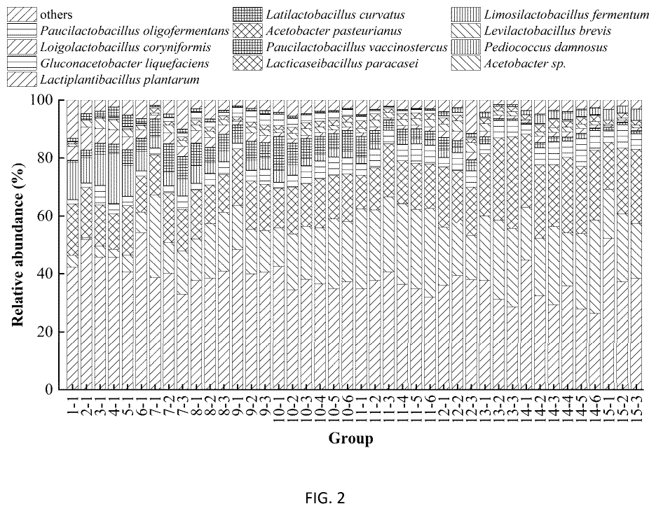

Species annotation was performed according to OUT classification results of the samples to further analyze changes of different fermentation types of microorganisms with the average relative abundance being greater than 1% in the fermentation process, and the results were shown in FIG. 2. It was found in results that main microorganisms in 15 generations belonged to Bacillota and Proteobacteria; and at the species level, L. plantarum, Lacticaseibacillus paracasei, Paucilactobacillus vaccinostercus, Loigolactobacillus coryniformis, L. brevis, Paucilactobacillus oligofermentans, L. fermentum, and L. curvatus are main dominant Lactobacillus, and moreover, Acetobacter, Gluconacetobacter liquefaciens, and Pediococcus damnosus are also main dominant microorganisms. The L. plantarum is the leading microorganisms, with the relative abundance reaching 30% or more, and reaching the highest 54.2% in the 6^{th} generation. Acetobacter grew persistently in the passaging process, reaching the highest in the 11th generation, with the relative abundance being 28.45%. The total change amplitude of Lactobacillus paracasei was not significant, with the relative abundance reaching 11.77%-25.16%.

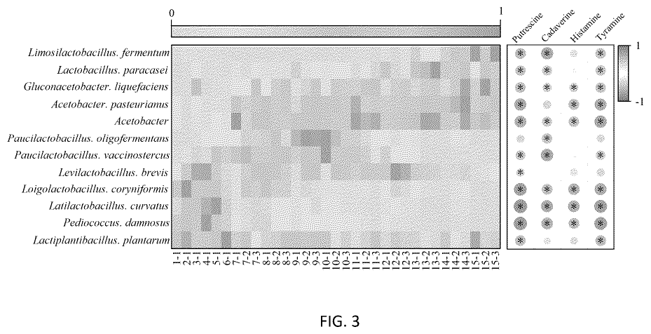

By observing the Spearman rank correlation between the microbial flora structure and the biogenic amines in the passaging process of the rice steeping microorganisms, as shown in FIG. 3, results showed that the L. plantarum, the P. damnosus, the L. curvatus, the L. coryniformis, and the L. brevis were negatively correlated to the content of the biogenic amines. Although the above microorganisms prompted generation of cadaverine, the concentration of cadaverine in the rice steeping water in the sample was less than 1 mg/L, which had less contribution to the content of the total biogenic amines; the Acetobacter, the Acetobacter pasteurianus, the G. liquefaciens, the L. paracasei, and the L. fermentum significantly promoted formation of the putrescine, histamine, and tyramine. The putrescine and tyramine are the leading biogenic amines in the rice steeping water, contributions of which to the content of the total biogenic amines would reach 90% or more.

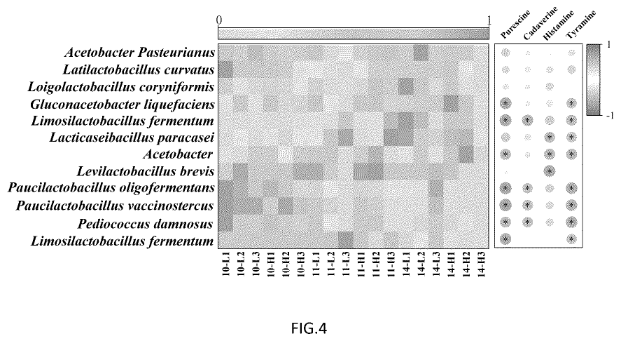

It was found by observing FIG. 1 that the spans of the content of the biogenic amines in the 10^{th}, 11^{th}, and 14^{th} generations were large, and differences of the highest and lowest biogenic amine flora structures in the 3 generations were analyzed, with results shown in FIG. 4. The L. plantarum and the Acetobacter were the main differential microorganisms, and difference values of relative abundance were both about 10%. The relative abundance of the Acetobacter in the group with the high content of the biogenic amines was significantly higher than that of the group with the low content of the biogenic amines. The difference value of the relative abundance was 8.9%-19.4%. With increase of the relative abundance of the Acetobacter and a decrease of the relative abundance of the L. plantarum, the biogenic amines were increased gradually. The Spearman rank correlation between the microbial flora structure and the biogenic amines showed that the L. plantarum, the P. vaccinostercus, the L. coryniformis, and the P. damnosus significantly inhibited the generation of putrescine and tyramine (p<0.05), the L. curvatus and the L. brevis had a certain inhibitory effect to putrescine and tyramine and were main biogenic amine degrading microorganisms; and the Acetobacter, the G. liquefaciens, the L. fermentum, and the L. paracasei significantly promoted generation of the biogenic amines (p<0.05).

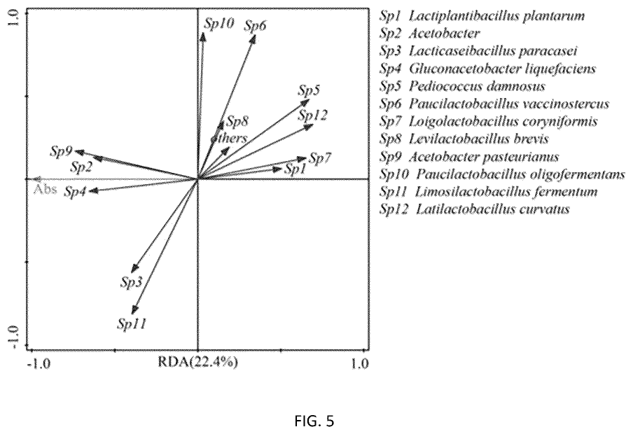

In order to understand the relation between the dominant microbial flora and the biogenic amines in the artificial selective enrichment experiment, redundancy analysis was performed on the dominant microbial flora and the biogenic amines, as shown in FIG. 5. Results indicated that the L. plantarum, the P. damnosus, the P. vaccinostercus, the L. coryniformis, the Bre, the P. oligofermentans, and the L. curvatus were negatively correlated to the content of the biogenic amines. Although the relative abundances of the L. coryniformis, the P. oligofermentans, and the L. curvatus were about 1%, the biogenic amines were significantly inhibited. The Acetobacter, the L. paracasei, the G. liquefaciens, the A. pasteurianus, and the L. fermentum significantly promoted the generation of the main biogenic amines.

To sum up, the results showed that the L. plantarum, the P. damnosus, the L. curvatus, the L. coryniformis, and the L. curvatus were significantly correlated to the reduction of the biogenic amines in the rice steeping water; and the Acetobacter, the L. paracasei, the G. liquefaciens, the A. pasteurianus, and the L. fermentum significantly promoted the generation of the biogenic amines.

### Example 2: Screening and Recombination of a Biogenic Amine Degrading Core Microbial Flora in Rice Steeping Water

It was found by successional combination with the content of the biogenic amines in the microecological environment of the rice steeping water in example 1 that the L. plantarum, the P. damnosus, the L. curvatus, the L. coryniformis, and the L. brevis were significantly correlated to the reduction of the biogenic amines in the rice steeping water. 31 L. plantarum (respectively named as L-3, L-8, L-13, L-14, L-16, L-17, L-18, L-21, L-23, L-24, L-26, L-27, L-28, L-33, L-34, L-35, L-36, L-40, L-41, L-42, L-43, L-45, L-47, L-48, L-49, L-51, L-52, L-53, L-54, L-55, and L-57), 3 L. brevis (respectively named as L-9, L-12, and L-15), and 1 L. curvatus (MG-28) were separated therefrom, and the above cultures (table 1) did not generate the biogenic amines. The above lactic acid bacteria were combined, including one L. plantarum, one L. brevis, and one L. curvatus (MG-28) assembled into a ternary interacting lactic acid bacteria flora, totally 93 combinations (table 1). A microbial flora model with the best effect of inhibiting invasion of the amine producing microorganisms was screened.

Specific steps are as follows:

### 1. Activation of a Strain

The lactic acid bacteria screened in table 1 were taken and activated for the first time on a solid MRS culture medium, a single colony was picked and the single colony was cultured in a liquid MRS culture medium at 37° C. for 24 h, after the second activation, the lactic acid bacteria were inoculated into a saccharified liquid with an inoculum size of 1% (v/v), and the lactic acid bacteria were cultured at 37° C. for 48 h to complete third activation.

### 2. Inoculation of Lactic Acid Bacteria and Plant Rice Steeping Water in a Rice Steeping Process

Glutinous rice was placed in a beaker, and water was added at a solid-liquid ratio of 1:1.2 to obtain rice steeping water; an activated L. plantarum, L. brevis, and L. curvatus (MG-28) bacteria solution were inoculated into the rice steeping water with an inoculum size of volume fraction 3% (the volume ratio is 1:1:1), with the total inoculum size being 9%. Contract had been made with clean water rice steeping without inoculation and singly inoculated L. plantarum L1901. Finally, plant rice steeping water accounting for 1% in volume of total rice steeping water was added into each group, and the mixture was left stand for 2 d.

The biogenic amines in the rice steeping water were detected on the 2^{nd} day, and the results of the biogenic amines in the rice steeping water of 93 combinations of rice steeping in two days were shown in table 1.

TABLE 1
Simulation of plant rice steeping to inhibit
the generation of biogenic amines (mg/L)

| | L-9 | L-12 | L-15 |
|---|---|---|---|
| L-3 | 51.36 ± 5.14 | 52.78 ± 1.23 | 53.71 ± 5.10 |
| L-8 | 50.45 ± 0.78 | 49.10 ± 5.41 | 45.12 ± 1.23 |
| L-13 | 50.41 ± 0.36 | 52.12 ± 4.15 | 53.14 ± 1.24 |
| L-14 | 56.12 ± 3.21 | 54.12 ± 3.46 | 54.12 ± 3.17 |
| L-16 | 45.41 ± 3.12 | 46.22 ± 3.14 | 41.12 ± 6.12 |
| L-17 | 45.12 ± 6.13 | 41.12 ± 4.13 | 42.13 ± 1.23 |
| L-18 | 44.14 ± 2.10 | 47.12 ± 1.56 | 41.13 ± 4.12 |
| L-21 | 53.12 ± 4.14 | 51.24 ± 9.10 | 56.12 ± 1.23 |
| L-23 | 60.12 ± 2.30 | 59.12 ± 1.20 | 60.12 ± 1.23 |
| L-24 | 52.12 ± 5.41 | 51.02 ± 6.12 | 53.12 ± 2.10 |
| L-26 | 55.12 ± 5.10 | 54.12 ± 3.10 | 56.12 ± 3.21 |
| L-27 | 40.15 ± 5.41 | 40.20 ± 5.14 | 43.12 ± 0.78 |
| L-28 | 59.12 ± 4.78 | 56.12 ± 2.41 | 57.12 ± 4.12 |
| L-33 | 59.13 ± 4.10 | 58.12 ± 1.23 | 52.14 ± 8.12 |
| L-34 | 60.12 ± 7.12 | 64.12 ± 1.20 | 64.12 ± 4.78 |
| L-35 | 61.20 ± 3.12 | 65.02 ± 2.20 | 65.12 ± 4.12 |
| L-36 | 63.12 ± 4.17 | 66.12 ± 8.14 | 61.78 ± 3.14 |
| L-40 | 65.14 ± 2.10 | 60.12 ± 2.47 | 64.12 ± 6.87 |
| L-41 | 45.12 ± 2.13 | 62.14 ± 3.56 | 63.13 ± 2.13 |
| L-42 | 67.12 ± 3.12 | 65.12 ± 4.32 | 62.13 ± 4.12 |
| L-43 | 45.12 ± 7.12 | 46.32 ± 3.10 | 41.63 ± 1.02 |
| L-45 | 42.13 ± 2.13 | 43.32 ± 2.10 | 42.13 ± 0.23 |
| L-47 | 45.12 ± 0.89 | 46.12 ± 0.65 | 47.14 ± 1.23 |
| L-48 | 39.01 ± 0.74 | 41.23 ± 0.74 | 42.48 ± 1.32 |
| L-49 | 48.01 ± 3.20 | 46.12 ± 4.12 | 47.12 ± 2.03 |
| L-51 | 41.02 ± 7.32 | 41.56 ± 1.23 | 46.12 ± 0.78 |
| L-52 | 41.89 ± 7.23 | 42.13 ± 2.10 | 42.30 ± 0.41 |
| L-53 | 38.54 ± 2.40 | 40.54 ± 4.15 | 42.15 ± 0.78 |
| L-54 | 39.30 ± 4.16 | 40.56 ± 1.23 | 40.15 ± 4.12 |
| L-55 | 45.07 ± 0.75 | 47.41 ± 2.13 | 40.12 ± 4.32 |
| L-57 | 40.13 ± 0.78 | 40.37 ± 0.15 | 40.31 ± 4.32 |

Remarks: the first line is L. brevis, the first row is L. plantarum, and the intersection of the horizontal rows represents generations of the biogenic amines after the two bacteria and L. curvatus are respectively added into 9% plant rice steeping water at a proportion of 3%.

The inhibitory effects of the combination of the L. plantarum L-53, L. brevis L-9, and L. curvatus MG-28 on the amine producing microorganisms were the most significant, and the content of the biogenic amines after rice steeping in two days was 38.54±0.21 mg/L.

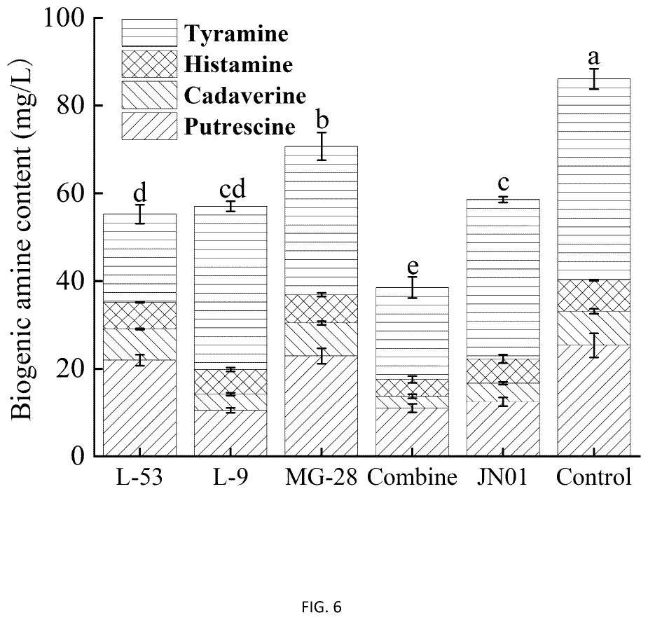

After detection, the content of the biogenic amines in the single L. plantarum L1901 was 58.31±0.35 mg/L, the content of the biogenic amines in the clean water control group was 113.81±2.41 mg/L (as shown in FIG. 6), and compared with the single L. plantarum L1901 and the clean water control group, the content of the biogenic amines in the ternary microbial combination (L. plantarum L-53, L. brevis L-9, and L. curvatus MG-28 were inoculated at a proportion of 1:1:1) were respectively reduced by 33.90% and 66.14%. Compared with the single L. plantarum L-53, the single L. brevis L-9, and the single L. curvatus MG-28, the combination (L. plantarum L-53, L. brevis L-9, and L. curvatus MG-28 were inoculated at a proportion of 1:1:1) were respectively reduced by 32.23%, 49.79%, and 57.95%.

Results showed that compared with single bacterial pure rice steeping, a ternary lactic acid bacteria interacting model (L. plantarum L-53, L. brevis L-9, and L. curvatus MG-28 were inoculated at a proportion of 1:1:1) had a better infectious microbe interference resisting ability, and the inhibition ratio of the biogenic amines was increased by 33.90%. Therefore, L. plantarum L-53, L. brevis L-9, and L. curvatus MG-28 were selected as target strains. L. plantarum L-53, L. brevis L-9, and L. curvatus MG-28 were respectively preserved in China Center for Type Culture Collection on Sep. 25, 2023, with the preservation address: Wuhan University, Wuhan, China.

### Example 3: Inhibition of Plant Cyclic Rice Steeping on Biogenic Amine Producing Microorganisms

### 1. Activation of a Strain

L. plantarum L-53, L. brevis L-9, and L. curvatus MG-28 were activated for the first time on a solid MRS culture medium, a single colony was picked and the single colony was cultured in a liquid MRS culture medium at 37° C. for 24 h, after second activation, the lactic acid bacteria were inoculated into a saccharified liquid with an inoculum size of 1% and cultured at 37° C. for 48 h to complete third activation, and L. plantarum L-53, L. brevis L-9, and L. curvatus MG-28 bacteria solutions were respectively prepared.

### 2. Simulation of Cyclic Rice Steeping in a Plant Environment

Glutinous rice was placed in a beaker, water was added at a solid-liquid ratio of 1:1.2, the activated L. plantarum L-53 bacteria solution, the L. brevis L-9 bacteria solution, and the L. curvatus MG-28 bacteria solution were respectively inoculated into the rice steeping water at the inoculum size of volume fraction 3%, the total inoculum size being 9%. Contract had been made with clean water rice steeping without inoculation and singly inoculated L. plantarum L1901. In a room temperature condition, after rice steeping for 2 d, the rice steeping water was collected, and clean water was replenished to achieve an initial water volume to obtain rice steeping water-1;

Glutinous rice was placed in a beaker, and the rice steeping water-1 (plant rice steeping water accounting for 1% of the initial total lactic acid bacteria inoculum size was added into the rice steeping water-1) was added at a solid-liquid ratio of 1:1.2 without inoculating lactic acid bacteria again for continuous rice steeping. Contract had been made with clean water rice steeping without inoculation and singly inoculated L. plantarum L1901. After rice steeping for 2 d, the rice steeping water was collected, and clean water was replenished to achieve an initial water volume to obtain rice steeping water-2; and after rice steeping for 2 d every time, cycles were performed 2 times according to the above method.

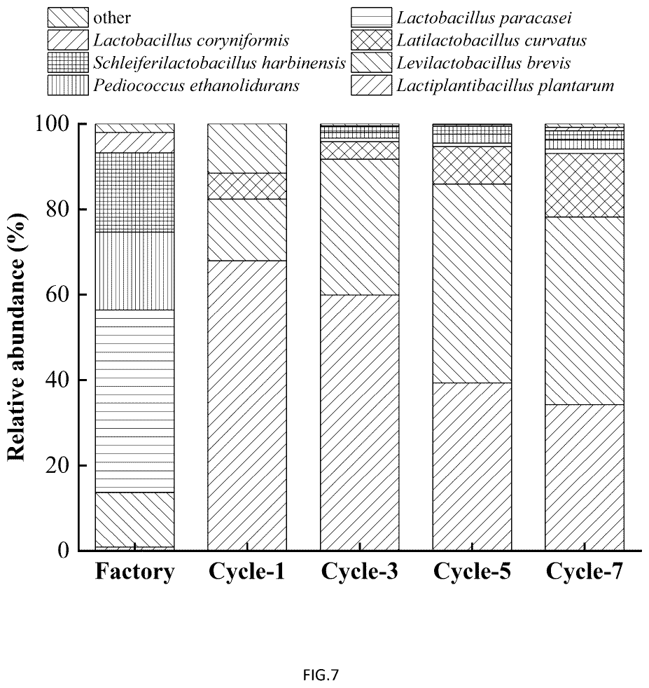

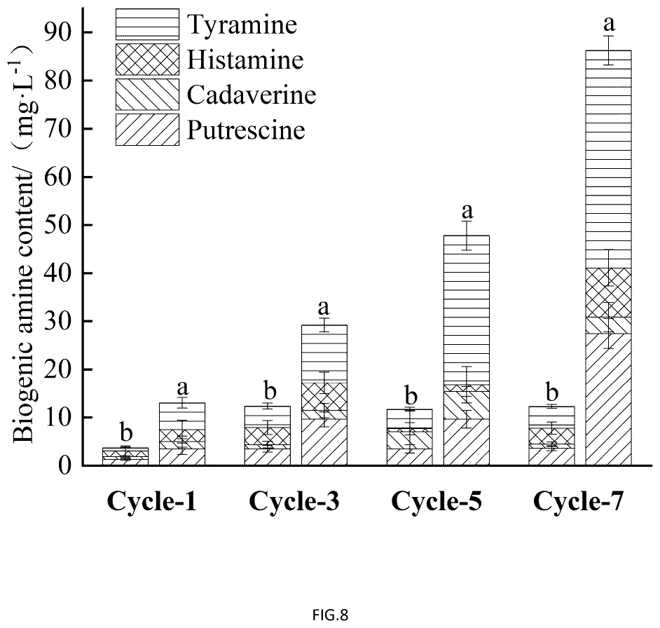

The cyclic modes of the rice steeping water for the group inoculated with the L. plantarum L1901 and the clean water rice steeping water control group without inoculation of lactic acid bacteria were the same as that of the inoculated group. Moreover, in the initial period of cyclic rice steeping every time, the plant rice steeping water accounting for 1% of the initial total lactic acid bacteria inoculum size was added to stimulate a plant rice steeping environment to observe the inhibitory effect on the amine producing microorganisms in the cyclic rice steeping process. The content of the biogenic amines in the rice steeping water was recorded in the cyclic process, the specific generation condition of the biogenic amines was shown in table 2, and drawing results were shown in FIG. 7 and FIG. 8.

TABLE 2
Contents of the biogenic amines in the rice steeping water obtained
in different groups in different cycle numbers (mg/L)

| | Cycle | | | | | |
|---|---|---|---|---|---|---|
| | number | Putrescine | Cadaverine | Histamine | Tyramine | Total biogenic amines |
| Ternary | 1 | 2.05 | 0.67 | 1.03 | 2.38 | 6.13 ± 2.83 |
| composition | 2 | 1.84 | 0.3 | 1.88 | 11.33 | 15.35 ± 1.70 |
| | 3 | 2.38 | 0.29 | 3.25 | 19.18 | 25.1 ± 3.99 |
| | 4 | 1.72 | 0.54 | 4.81 | 42.64 | 49.71 ± 2.32 |
| L1901 | 1 | 4.01 | 1.29 | 2.13 | 4.02 | 11.75 ± 1.83 |
| | 2 | 15.01 | 4.56 | 6.18 | 20.36 | 46.11 ± 1.35 |
| | 3 | 21.25 | 5.56 | 7.36 | 30.23 | 64.40 ± 3.37 |
| | 4 | 28.54 | 7.13 | 9.12 | 37.78 | 82.57 ± 2.62 |
| Clean | 1 | 25.16 | 0.78 | 4.62 | 27.38 | 58.24 ± 2.84 |
| water | 2 | 35.12 | 4.12 | 12.53 | 40.45 | 92.02 ± 2.83 |
| control | 3 | 40.12 | 4.52 | 13.60 | 47.12 | 105.36 ± 2.39 |
| group | 4 | 54.12 | 9.12 | 17.23 | 60.54 | 141.01 ± 2.11 |

Results showed that in the cyclic rice steeping process, the content of the biogenic amines was in a rising trend, and the rising speed of the ternary microorganism group was significantly lower than that of the control group and the L. plantarum L1901 group. After the 4^{th} cycle of the ternary microorganism group, the content of the biogenic amines was 49.1±2.33 mg/L, which was still lower than that after the 1st cycle in the control group (58.24±2.84 mg/L). The content of the biogenic amines in the L. plantarum L1901 group had reached 46.11±1.35 mg/L in the 2nd cycle. It was found by studies on types of the biogenic amines that the ternary microorganism group inhibited putrescine significantly, and the content of the biogenic amines after each cycle was lower than 3 mg/L.

### Example 4: Optimization of an Adding Proportion

### 1. Activation of a Strain

Lactic acid bacteria L. plantarum L-53, L. brevis L-9, and L. curvatus MG-28 screened were activated for the first time on a solid MRS culture medium respectively, a single colony was picked and the single colony was cultured in a liquid MRS culture medium at 37° C. for 24 h, the lactic acid bacteria were inoculated into a saccharified liquid with an inoculum size of 1% (v/v) and cultured at 37° C. for 48 h to complete third activation, so as to respectively prepare an L. plantarum L-53 bacteria solution, an L. brevis L-9 bacteria solution, and an L. curvatus MG-28 bacteria solution.

### 2. Optimization of an Adding Proportion

Glutinous rice was placed in a beaker, and water was added at a solid-liquid ratio of 1:1.2, an activated L. plantarum bacteria solution, L. brevis bacteria solution, and L. curvatus bacteria solution bacteria solution were respectively inoculated into the rice steeping water at inoculum sizes of volume fractions 1-7%, with the total inoculum size being 9% (a specific inoculum size shown in table 3). Contract had been made with the rice steeping water in the group inoculated with the L. plantarum L1901 and the clean water rice steeping control group without inoculation of the lactic acid bacteria. Finally, plant rice steeping water accounting for 10% of the total lactic acid bacteria inoculum size was added into each group. In a room temperature condition, the rice steeping water was collected after rice steeping for 2 d. The biogenic amines in the rice steeping water were detected on the second day, and the biogenic amine generation results after rice steeping for 2 d were shown in table 3.

TABLE 3
Inhibition of lactic acid bacteria at different adding
proportions on biogenic amine producing microorganisms

| | | | Content of biogenic |
|---|---|---|---|
| L-9 | L-53 | MG-28 | amines (mg/L) |
| 1% | 7% | 1% | 36.52 ± 5.14 |
| | 6% | 2% | 36.66 ± 2.13 |
| | 5% | 3% | 39.09 ± 3.14 |
| | 4% | 4% | 44.27 ± 4.16 |
| | 3% | 5% | 55.40 ± 2.43 |
| | 2% | 6% | 56.28 ± 2.35 |
| | 1% | 7% | 81.93 ± 0.12 |
| 2% | 6% | 1% | 33.90 ± 0.13 |
| | 5% | 2% | 40.79 ± 2.10 |
| | 4% | 3% | 42.40 ± 4.13 |
| | 3% | 4% | 53.28 ± 3.19 |
| | 2% | 5% | 51.62 ± 1.46 |
| | 1% | 6% | 67.47 ± 3.17 |
| 3% | 5% | 1% | 38.14 ± 2.18 |
| | 4% | 2% | 38.33 ± 1.26 |
| | 3% | 3% | 46.10 ± 1.39 |
| | 2% | 4% | 48.18 ± 1.46 |
| | 1% | 5% | 73.27 ± 1.02 |
| 4% | 4% | 1% | 34.48 ± 1.78 |
| | 3% | 2% | 38.14 ± 4.10 |
| | 2% | 3% | 50.35 ± 1.78 |
| | 1% | 4% | 60.24 ± 3.17 |
| 5% | 3% | 1% | 37.00 ± 1.23 |
| | 2% | 2% | 60.68 ± 2.18 |
| | 1% | 3% | 59.80 ± 3.91 |
| 6% | 2% | 1% | 47.21 ± 4.32 |
| | 1% | 2% | 68.68 ± 0.17 |
| 7% | 1% | 1% | 65.10 ± 1.07 |

| | |
|---|---|
| L1901 | 63.10 ± 1.07 |
| con | 113.80 ± 1.03 |

Remark: con was the clean water control group where the plant rice steeping water was only processed.

### Results Show:

(1) in a condition of the inoculum size with the total amount being 9%, when the adding amount of L. plantarum L-53 is greater than 1%, the L. brevis bacteria solution and the L. curvatus bacteria solution are added at any proportion, which can realize the technical effect of the disclosure;

(2) When L. plantarum: L. brevis: L. curvatus=6:2:1, the inhibitory effect on the biogenic amines is the strongest, and finally, the content of the biogenic amines is 33.90±0.13 mg/L, which, compared with that in the control group where the plant rice steeping water is only processed, is reduced by 70.21%.

(3) When L. plantarum: L. brevis: L. curvatus=6:2:1, finally, the content of the biogenic amines is 33.90±0.13 mg/L, and in the same condition, when the L. plantarum, L. brevi, and L. curvatus are added at an equal proportion, the content of the biogenic amines is 46.10±1.39 mg/L, and the biogenic amine inhibitory effect is increased by 26.46%.

(4) When L. plantarum: (L. brevis+L. curvatus) is ≥2:7 (addition of the two bacteria refers to the total volume to be inoculated of the L. brevis and the L. curvatus after activation), the L. brevis bacteria solution and the L. curvatus bacteria solution are added at any proportion, which can realize the technical effect of the disclosure.

(5) When L. brevis: (L. plantarum+L. curvatus) is 4-5:5-4 (addition of the two bacteria refers to the total volume to be inoculated of the L. plantarum and the L. curvatus after activation), the L. plantarum bacteria solution and the L. curvatus bacteria solution are added at any proportion, which can realize the technical effect of the disclosure.

### Example 5: Optimization of a Cyclic Rice Steeping Process

When invasion of amine producing microorganisms was found in the early stage of a lab, the content of the biogenic amines was decreased as the acidity was raised. When the acidity reached 14 g/L, the acidity completely inhibited the amine producing microorganisms, so that zero growth of the biogenic amines was realized. Therefore, a novel cyclic rice steeping process was invented to better inhibit the growth and metabolism of the amine producing microorganisms.

### 1. Activation of a Strain

Lactic acid bacteria L. plantarum L-53, L. brevis L-9, and L. curvatus MG-28 screened were activated for the first time on a solid MRS culture medium respectively, a single colony was picked and the single colony was cultured in a liquid MRS culture medium at 37° C. for 24 h, the lactic acid bacteria were inoculated into a saccharified liquid with an inoculum size of 1% (v/v) and cultured at 37° C. for 48 h to complete third activation, so as to respectively prepare an L. plantarum L-53 bacteria solution, an L. brevis L-9 bacteria solution, and an L. curvatus MG-28 bacteria solution.

### 2. Optimization of the Process

Glutinous rice was placed in a beaker, water was added at a solid-liquid ratio of 1:1.2, the activated L. plantarum bacteria solution, the L. brevis bacteria solution, and the L. curvatus bacteria solution were respectively inoculated into the rice steeping water with inoculum sizes of volume fractions 6%, 2%, and 1%, with the total inoculum size being 9%. In a room temperature condition, the mixture was left to stand to ferment for 24 h, after 13%-20% of clean water was replenished, fermentation was continuously performed for 16 h in the room temperature condition, after 13%-20% of clean water was replenished, fermentation was continuously performed for 8 h in the room temperature condition, and after 13%-20% of clean water was replenished, fermentation was continuously performed for 48 h in the room temperature condition, all rice milk was collected for recycling, and in the whole rice steeping process, the mixture was uniformly stirred at every 8 h. Rice steeping water-1 was obtained;

The rice steeping water-1 was collected for a next round of rice steeping, glutinous rice was placed in a beaker, the rice steeping water-1 was added at a solid-liquid ratio of 1:1.2, plant rice steeping water accounting for 10% in total volume of the rice steeping water-1 was added in the initial rice steeping period to observe the inhibition condition of amine producing infectious microbes without inoculating the lactic acid bacteria, fermentation was performed for 8 h in the room temperature condition, after 13%-20% of clean water was replenished, fermentation was continuously performed for 8 h in the room temperature condition, after 13%-20% of clean water was replenished, fermentation was continuously performed for 8 h in the room temperature condition, after 13%-20% of clean water was replenished, fermentation was continuously performed for 24 h in the room temperature condition finally, all rice milk was collected for recycling, and in the whole rice steeping process, the rice milk was uniformly stirred at every 8 h. The above cyclic steps were cycled subsequently, and total acid changes of the rice steeping water in the cyclic process and the contents of the biogenic amines after the cyclic rice steeping was finished were recorded.

TABLE 4
Changes of the biogenic amines in an
improved cyclic rice steeping process

| | Content of biogenic | Content of control | Content of clean |
|---|---|---|---|
| | amines in ternary | L1901 immersed | water immersed |
| Cycle | composition | biogenic amines | biogenic amines |
| number | (mg/L) | (mg/L) | (mg/L) |
| 1 | 5.34 ± 0.22 | 13.04 ± 1.09 | 60.14 ± 2.16 |
| 2 | 4.96 ± 0.62 | 15.21 ± 0.69 | 70.11 ± 3.99 |
| 3 | 6.32 ± 1.34 | 29.21 ± 1.43 | 92.01 ± 3.12 |
| 4 | 5.23 ± 1.03 | 59.01 ± 3.02 | 116.11 ± 2.03 |

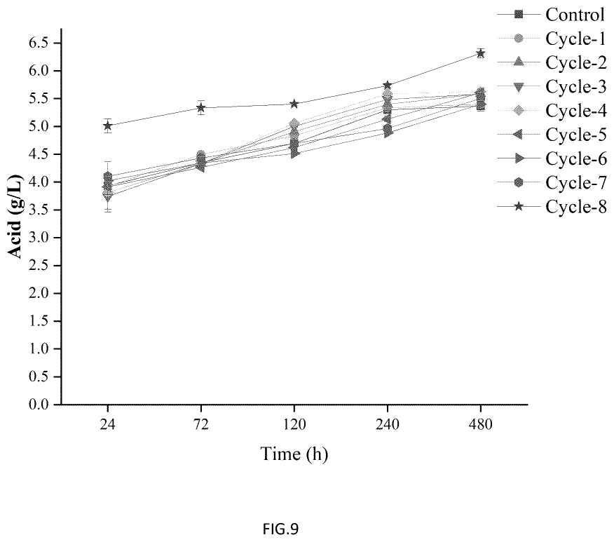

Results showed that as shown in FIG. 9, the acidity on the 3^{rd} day in the first cycle reached the maximum value, and subsequent cycles were substantially finished within 2 d, so that the rice steeping time was greatly shortened. Moreover, in the four cycles, there were no large fluctuations, which met the condition of recycling of the rice steeping water. A determination result of the biogenic amines after each cyclic rice steeping was finished showed, as shown in table 4, the biogenic amines were basically kept at a low level, which realized zero growth of the biogenic amines. In the process of simulating the infectious microbe interference resisting process in the plant environment, the biogenic amines of the L1901 rose continuously. The biogenic amines in the 4^{th} cycle increased to 59.01±3.02 mg/L, and the content of the biogenic amines was 11 times that in the disclosure.

3. Rice after Rice Steeping is Used to Brew Huangjiu, and Indexes of the Product are Detected as Follows:

sensory indexes: the Huangjiu is brown in color, clear, transparent and glossy, has elegant and mellow features, and is peculiar smell-free, mellow in taste, tasty and harmonious in body.

Physical and chemical indicators: content of total sugars≤15 g/L, non-sugar solids≥25 g/L, alcoholic strength≥15% vol, amino acid nitrogen≥0.4 g/L, pH value 3.8-4.0, content of calcium oxide≤0.8 g/L, and content of benzoic acid≤0.03 g/kg.

The indexes all meet the national standard. Compared with the control group 1 inoculated with L1901, the content of biogenic amines is decreased by 30%-50%, and compared with the control group 2 without inoculation, the content of biogenic amines is decreased by 50%-90%.

### Example 6: Addition of L. plantarum, L. Brevis, and L. curvatus in a Cyclic Rice Steeping Process of Table Vinegar to Inhibit Amine Producing Microorganisms

### 1. Activation of a Strain

The lactic acid bacteria screened were taken and activated for the first time on a solid MRS culture medium, a single colony was picked and the single colony was cultured in a liquid MRS culture medium at 37° C. for 24 h, the lactic acid bacteria were inoculated into a saccharified liquid with an inoculum size of 1%, and the lactic acid bacteria were cultured at 37° C. for 48 h to complete third activation.

### 2. Optimization of an Adding Proportion

Sorghum, glutinous rice, and rice were crushed at a proportion of 4:3:3, subjected to a 40-mesh screen, and placed in a beaker. Water was added with a solid-liquid ratio of 1:1.2. The activated L. plantarum, L. brevis, and L. curvatus were respectively inoculated into the rice steeping water with inoculum sizes of volume fractions 6%, 2%, and 1%, with the total inoculum size being 9%. 13%-20% (rice to water ratio) of clean water was replenished in batches in a fast acid-raising period (in an exponential growth phase, the process might refer to example 5), totally 40%-60% of clean water was added. The mixture was stirred once at every 8 h, and rice steeping was stopped till the acidity was no longer raised. Rice milk was collected for a next round of rice steeping. 10% of plant rice steeping water was added in the initial rice steeping stage without inoculating the lactic acid bacteria again. The above adding and stirring steps were repeated. The contents of the biogenic amines after the cycle was finished were recorded, and results were shown in table 5.

TABLE 5
Changes of the biogenic amines in the cyclic rice steeping process

| Cycle | Ternary | Control L1901 | Clean water |
|---|---|---|---|
| number | composition (mg/L) | immersed (mg/L) | immersed (mg/L) |
| 1 | 6.24 ± 0.12 | 12.14 ± 2.09 | 57.14 ± 4.16 |
| 2 | 5.66 ± 0.32 | 17.21 ± 0.39 | 69.01 ± 2.99 |
| 3 | 5.42 ± 2.14 | 30.21 ± 2.43 | 92.31 ± 4.12 |
| 4 | 5.22 ± 2.13 | 60.01 ± 4.32 | 126.11 ± 2.83 |

Rice after rice steeping is used to make table vinegar, and indexes of the product are detected as follows:

Sensory indexes: the product is amber in color, soft in sour, lingering in afterwaste and peculiar smell-free, has a unique flavor of table vinegar, and is clear in body.

Physical and chemical indicators: total acid≥3.50 g/100 mL, nonvolatile acids≥0.6 g/100 mL, soluble salt-free solids≥0.7 g/100 mL.

Compared with the control group 1 inoculated with L1901, the content of biogenic amines is decreased by 20%-40%, and compared with the control group 2 without inoculation, the content of biogenic amines is decreased by 40%-80%.

### Example 7: Addition of L. plantarum, L. Brevis, and L. curvatus in a Cyclic Rice Steeping Process of Cooking Wine to Inhibit Amine Producing Microorganisms

### 1. Activation of a Strain

The lactic acid bacteria screened were taken and activated for the first time on a solid MRS culture medium, a single colony was picked and the single colony was cultured in a liquid MRS culture medium at 37° C. for 24 h, the lactic acid bacteria were inoculated into a saccharified liquid with an inoculum size of 1%, and the lactic acid bacteria were cultured at 37° C. for 48 h to complete third activation.

### 2. Optimization of an Adding Proportion

Polished round-grained rice was placed in a beaker. Water was added with a solid-liquid ratio of 1:1.2. The activated L. plantarum, L. brevis, and L. curvatus were respectively inoculated into the rice steeping water with inoculum sizes of volume fractions 6%, 2%, and 1%, with the total inoculum size being 9%. 13%-20% (rice to water ratio) of clean water was replenished in batches in a fast acid-raising period (in an exponential growth phase, the process might refer to example 5), totally 40%-60% of clean water was added. The mixture was stirred once at every 8 h, and rice steeping was stopped till the acidity was no longer raised. Rice milk was collected for a next round of rice steeping. 10% of plant rice steeping water was added in the initial rice steeping stage without inoculating the lactic acid bacteria again. The above adding and stirring steps were repeated. The contents of the biogenic amines after the cycle was finished were recorded, and results were shown in table 6.

TABLE 6
Changes of the biogenic amines in the cyclic rice steeping process

| Cycle | Ternary | Control L1901 | Clean water |
|---|---|---|---|
| number | composition (mg/L) | immersed (mg/L) | immersed (mg/L) |
| 1 | 5.14 ± 0.02 | 11.14 ± 0.02 | 58.14 ± 0.10 |
| 2 | 6.16 ± 0.22 | 18.21 ± 1.36 | 79.01 ± 1.96 |
| 3 | 4.42 ± 0.14 | 35.21 ± 3.46 | 99.31 ± 3.10 |
| 4 | 5.22 ± 1.13 | 65.01 ± 1.37 | 120.11 ± 0.88 |

Rice after rice steeping is used to make cooking wine, and indexes of the product are detected as follows:

Sensory indexes: the cooking wine is light yellow to rufous in color and glossy, has unique mellowness of cooking wine, is harmonious in fragrance, pure in taste, peculiar smell-free, and clear and transparent.

Physical and chemical indicators: alcoholic strength≥12.0% vol, amino acid nitrogen≥0.5 g/L, total acid≤5.0 g/L, content of table salt≥10.0 g/L.

Compared with the control group 1 inoculated with L1901, the content of biogenic amines is decreased by 25%-45%, and compared with the control group 2 without inoculation, the content of biogenic amines is decreased by 45%-85%.

### Example 8: Addition of L. plantarum, L. Brevis, and L. curvatus in a Cyclic Rice Steeping Process of Rice Noodles to Inhibit Amine Producing Microorganisms

### 1. Activation of a Strain

The lactic acid bacteria screened were taken and activated for the first time on a solid MRS culture medium, a single colony was picked and the single colony was cultured in a liquid MRS culture medium at 37° C. for 24 h, the lactic acid bacteria were inoculated into a saccharified liquid with an inoculum size of 1%, and the lactic acid bacteria were cultured at 37° C. for 48 h to complete third activation.

### 2. Optimization of an Adding Proportion

Rice was placed in a beaker. Water was added with a solid-liquid ratio of 1:1.8. The activated L. plantarum, L. brevis, and L. curvatus were respectively inoculated into the rice steeping water with inoculum sizes of volume fractions 6%, 2%, and 1%, with the total inoculum size being 9%. The rice was soaked for 16 h. Rice milk was collected for a next round of rice steeping. 10% of plant rice steeping water was added in the initial rice steeping stage and 50% of clean water was replenished without inoculating the lactic acid bacteria again. The above adding and stirring steps were repeated. The contents of the biogenic amines after the cycle was finished were recorded, and results were shown in table 7.

TABLE 7
Changes of the biogenic amines in the cyclic rice steeping process

| Cycle | Ternary | Control L1901 | Clean water |
|---|---|---|---|
| number | composition (mg/L) | immersed (mg/L) | immersed (mg/L) |
| 1 | 4.04 ± 0.42 | 5.14 ± 0.42 | 60.14 ± 0.92 |
| 2 | 10.06 ± 0.32 | 20.21 ± 2.36 | 100.01 ± 0.86 |
| 3 | 15.22 ± 0.54 | 60.21 ± 3.46 | 110.31 ± 3.36 |
| 4 | 19.92 ± 1.03 | 90.01 ± 2.03 | 120.11 ± 6.13 |

Rice after rice steeping is used to make rice noodles, and indexes of the product are detected as follows:

Sensory indexes: the rice flour block is uniform in color, soft in smell and peculiar smell-free, smooth in surface, smooth in taste, elastic, and not sticky.

Physical and chemical indicators: content of rice flour block≤12.0%, rehydration rate≥220%, short strip rate≤10.0%, strip sticking rate≤2.0%, acidity of rice flour block≤1.9° T, total arsenic content≤0.2 mg/kg, lead content≤0.3 mg/kg.

Compared with the control group 1 inoculated with L1901, the content of biogenic amines is decreased by 25%-45%, and compared with the control group 2 without inoculation, the content of biogenic amines is decreased by 45%-85%.

### Example 9: Addition of L. plantarum, L. Brevis, and L. curvatus in a Cyclic Rice Steeping Process of Soybeans to Inhibit Amine Producing Microorganisms

### 1. Activation of a Strain

The lactic acid bacteria screened were taken and activated for the first time on a solid MRS culture medium, a single colony was picked and the single colony was cultured in a liquid MRS culture medium at 37° C. for 24 h, the lactic acid bacteria were inoculated into a saccharified liquid with an inoculum size of 1%, and the lactic acid bacteria were cultured at 37° C. for 48 h to complete third activation.

### 2. Optimization of an Adding Proportion

Soybeans were placed in a beaker. Water was added with a solid-liquid ratio of 1:1.2. The activated L. plantarum, L. brevis, and L. curvatus were respectively inoculated into the rice steeping water with inoculum sizes of volume fractions 6%, 2%, and 1%, with the total inoculum size being 9%. 13%-20% (rice to water ratio) of clean water was replenished in batches in a fast acid-raising period (in an exponential growth phase, the process might refer to example 5), totally 40%-60% of clean water was added. The mixture was stirred once at every 8 h, and rice steeping was stopped till the acidity was no longer raised. Rice milk was collected for a next round of rice steeping. 10% of plant rice steeping water was added in the initial rice steeping stage without inoculating the lactic acid bacteria again. The above adding and stirring steps were repeated. The contents of the biogenic amines after the cycle was finished were recorded, and results were shown in table 8.

TABLE 8
Changes of the biogenic amines in the cyclic rice steeping process

| Cycle | Ternary | Control L1901 | Clean water |
|---|---|---|---|
| number | composition (mg/L) | immersed (mg/L) | immersed (mg/L) |
| 1 | 4.14 ± 0.12 | 10.14 ± 0.42 | 60.14 ± 0.12 |
| 2 | 5.26 ± 0.32 | 19.21 ± 2.36 | 95.01 ± 0.86 |
| 3 | 6.42 ± 0.24 | 40.21 ± 3.46 | 103.31 ± 2.16 |
| 4 | 5.02 ± 0.13 | 80.01 ± 2.03 | 130.11 ± 2.87 |

For soybeans after rice steeping, indexes of the product are detected as follows:

Sensory indexes: the soybeans are smooth in surface and free of wrinkles. The sections are permeated without hard cores. The soybeans are white in color, smooth and fine, and elastic.

Compared with the control group 1 inoculated with L1901, the content of biogenic amines is decreased by 50% or more, and compared with the control group 2 without inoculation, the content of biogenic amines is decreased by 90% or more.

Although disclosed with preferred examples above, the disclosure is not limited by the examples. Any person skilled in the art may make various alternations and modifications without departing the spirit and scope of the disclosure. Therefore, the scope of protection of the disclosure should be subject to the scope of the disclosure as defined in the claims.

What is claimed is:

1. A composition, containing Lactiplantibacillus plantarum L-53 or fermentation liquor thereof, Levilactobacillus brevis L-9 or fermentation liquor thereof, and Latilactobacillus curvatus MG-28 or fermentation liquor thereof,
wherein the L. plantarum L-53 has been preserved in China Center for Type Culture Collection on Sep. 25, 2023, with preservation No. CCTCC NO: M 20231784;
the L. brevis L-9 has been preserved in China Center for Type Culture Collection on Sep. 25, 2023, with preservation No. CCTCC NO: M 20231786; and
the L. curvatus MG-28 has been preserved in China Center for Type Culture Collection on Sep. 25, 2023, with preservation No. CCTCC NO: M 20231785.

2. The composition according to claim 1, wherein an adding proportion of the L. plantarum in the composition is at least 23% based on viable count; an adding proportion of the L. brevis in the composition is at least 1-77% based on viable count; an adding proportion of the L. curvatus in the composition is at least 1-77% based on viable count; and the sum of the adding proportions of the L. plantarum, L. brevis, and L. curvatus is 100%.

3. A method for reducing a content of biogenic amines in a cereal product, wherein the composition according to claim 1 is added into a processing process of the cereal product.

4. The method according to claim 3, wherein the cereal product comprises any one or more of the following: Huangjiu (Chinese rice wine), Baijiu (Chinese liquor), table vinegar, rice vinegar, cooking wine, rice noodles, rice flour, and soybeans.

5. The method according to claim 3, wherein the composition is added into a rice steeping process of the cereal product.

6. The method according to claim 5, wherein rice steeping water added with the composition for rice steeping is recycled.

7. The method according to claim 6, wherein the recycling comprises: mixing the rice steeping water added with the composition for rice steeping with clear water in the proportion of 40-60% without inoculating the lactic acid bacteria again for a next round of rice steeping.

8. The method according to claim 3, wherein the rice steeping water added with the composition for rice steeping is recycled, and the recycling comprises: mixing the rice steeping water added with the composition for rice steeping with clear water in the proportion of 40-60% without inoculating the lactic acid bacteria again for a next round of rice steeping.

9. The method according to claim 8, comprising the following steps: 1) after rice steeping, adding the composition into the rice steeping water, and after static fermentation for 24-36 hours in a room temperature condition, replenishing 13%-20% of clean water; 2) after continuous fermentation for 8-24 hours in the room temperature condition, replenishing 13%-20% of clean water; 3) after continuous fermentation for 8-24 hours in the room temperature condition, replenishing 13%-20% of clean water; and 4) after continuous fermentation for 24-72 hours in the room temperature condition, collecting all rice milk for recycling, and stirring and uniformly mixing the rice milk at every 8-24 hours during recycling,
wherein the recycling comprises: steeping rice with all rice milk obtained in step 4), wherein the clean water is not replenished at this time and the lactic acid bacteria is not inoculated again; after rice steeping, performing fermentation for 8-24 hours in the room temperature condition and replenishing 13%-20% of clean water; performing continuous fermentation for 8-24 hours in the room temperature condition and replenishing 13%-20% of clean water; performing continuous fermentation for 8-24 hours in the room temperature condition and replenishing 13%-20% of clean water; performing continuous fermentation for 8-24 hours in the room temperature condition; and collecting all rice milk for the next round of rice steeping, and stirring and uniformly mixing the rice milk at every 8-24 hours during recycling.

10. The method according to claim 8, wherein the L. plantarum, the L. brevis, and the L. curvatus are activated before being inoculated, and the activating method is step-by-step extended cultivation according to an order of a solid plate, a triangular flask, and a cultivating pot, with specific operations as follows: performing first activation on the lactic acid bacteria on a solid MRS culture medium, picking a single colony and culturing the single colony in a liquid MRS culture medium at 28-37° C. for 12-24 hours, inoculating the lactic acid bacteria into a saccharified liquid with an inoculum size of 1%, and culturing the L. plantarum at 28-37° C. for 36-72 hours to complete third activation.
